(19) United States
(12) Patent Application Publication
(10) Pub. No.: US 2005/0053554 A1
(43) Pub. Date: Mar. 10, 2005

(54) Method for treatment of a region of the skin of a human subject, in particular cosmetic treatment by the trans-epidermal route using projection of a liquid under pressure

(76) Inventors: Jean-Pierre Devidal, Paris (FR); Jean-Pierre Michaud, Clamart (FR); Alain Corre, Paris (FR)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/500,868

(22) PCT Filed: Jan. 17, 2003
(86) PCT No.: PCT/FR03/00153

(30) Foreign Application Priority Data
Jan. 21, 2002 (FR) 02/00701

(51) Int. Cl. A61K 9/00; A61K 9/26

(52) U.S. Cl. 424/47

(57) ABSTRACT

The invention concerns a method for the treatment, in particular cosmetic, of an area of the skin of a human subject transepidermally, comprising the step consisting of spraying a pressurised liquid onto the area to be treated, by means of a spray nozzle (25). The invention also concerns a device for implementing such a method, as well as a hand piece for such a device.

The invention concerns a method for the treatment, in particular cosmetic, of an area of the skin of a human subject transepidermally, as well as a device for implementing such a method, and a hand piece for such a device.

Methods of rejuvenating the skin are already known, such as laser methods, chemical peelings, mesotherapy techniques, injections of collagen and other products, resorbable or not, or methods of abrading the skin by spraying abrasive powders.

However, these methods have several drawbacks: they are in fact liable to cause pain and/or secondary effects.

In addition they are usually socially incapacitating for the patients, since the skin, having been highly stressed, regains its normal appearance only after a significant lapse of time.

To mitigate these drawbacks, the invention proposes a method for the treatment, in particular cosmetic, of an area of the skin of a human subject transepidermally, intended in particular to allow the rejuvenation of the skin and/or improvement to imperfections of the skin.

Implementing this method makes it possible to make active substances penetrate through the epidermis, without causing any significant incapacitation. Thus, once the treatment is ended, the skin regains its normal appearance within a very short period of time, ranging from a few minutes to a few hours, and the patients can consequently resume their normal activities very rapidly.

In addition, this method requires no anaesthesia, and does not cause any pain in the patient. It makes it possible to make active products intended to improve particularly the appearance of the skin, for example hydration, penetrate deeply.

To this end, and according to a first aspect, the invention proposes a method for the treatment, in particular cosmetic, of an area of the skin of a human subject transepidermally, comprising the step consisting of spraying a liquid under pressure on the area to be treated, by means of a spray nozzle.

According to a second aspect, an object of the invention is a device for the treatment of an area of the skin of a human subject transepidermally, comprising a spray nozzle, and comprising means of pressurising a liquid, and at least one conduit disposed between the pressurising means and the spray nozzle.

According to a third aspect, the invention concerns a hand piece for such a device, comprising control means arranged so as, in the idle position of the said hand piece, to stop the pressurised liquid through the contact of the end of the piston on a piece and, in the position of use, to move away the said end of the piece, so as to release the liquid in order to supply the spray nozzle.

Other objects and advantages of the invention will emerge during the following description given with reference to the accompanying drawings, in which:

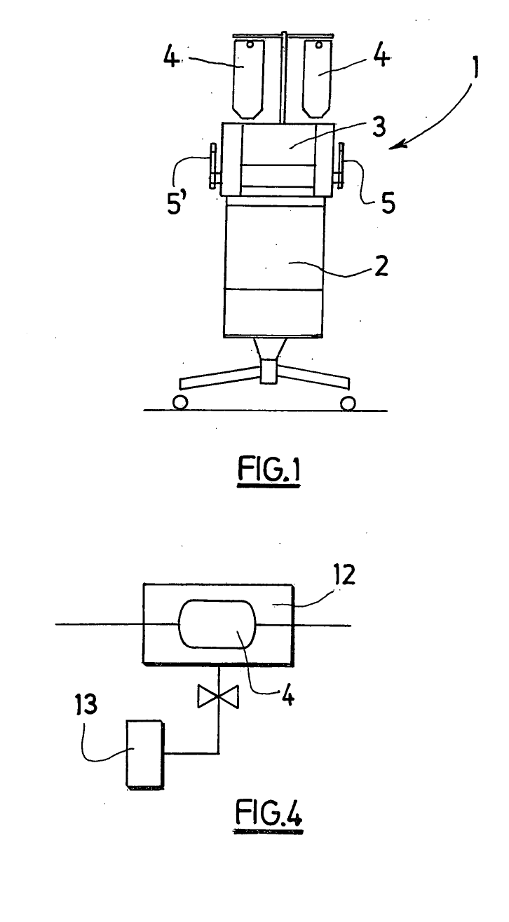

FIG. 1 is a schematic front view of a device for implementing the method according to the invention;

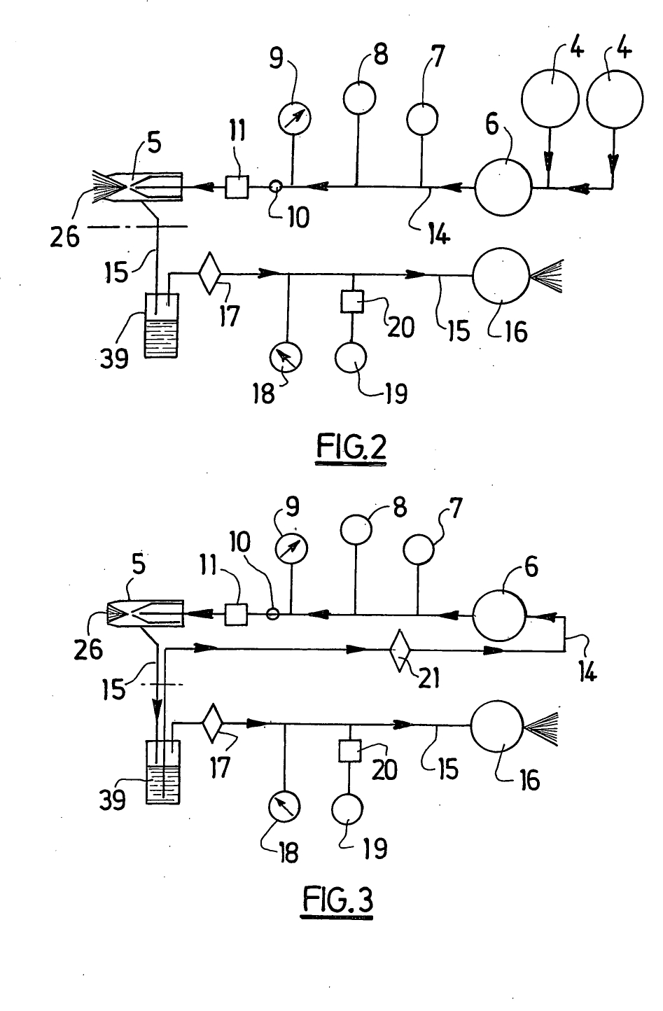

FIG. 2 is a schematic view of the elements forming the device of FIG. 1, according to a first embodiment in which the device functions in open circuit;

FIG. 3 is a schematic view of the elements forming the device of FIG. 1, according to a second embodiment in which the device functions in closed circuit;

FIG. 4 is a schematic view of the means of pressurising the device of FIG. 1 according to one embodiment;

FIG. 5 is a view in section of a hand piece forming part of the device of FIG. 1, according to a first embodiment, comprising a nozzle for spraying a pressurised liquid;

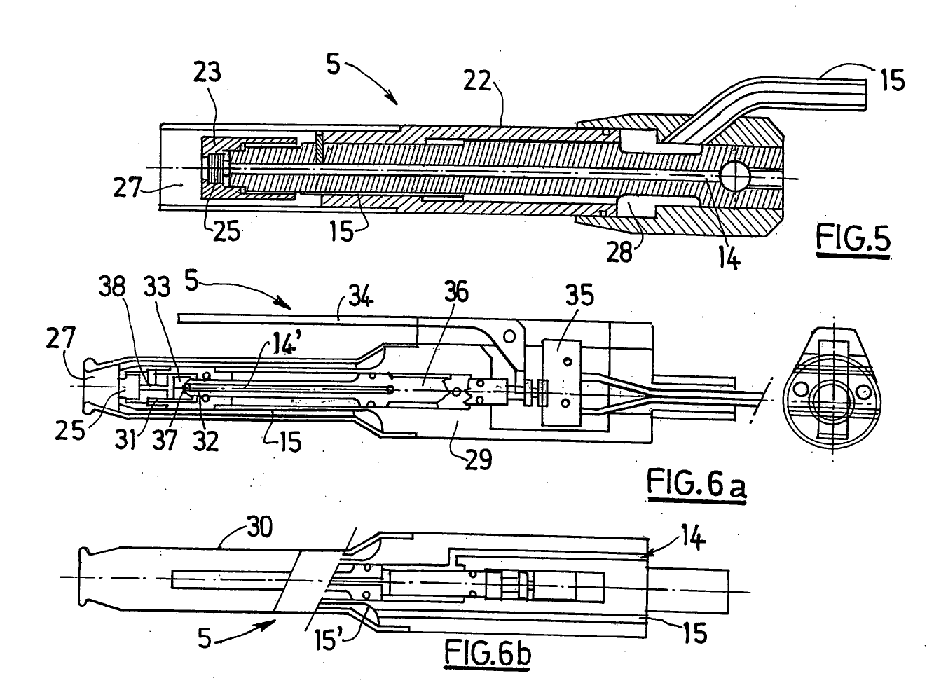

FIGS. 6a and 6b are respectively a view in section and a plan view of a hand piece forming part of the device of FIG. 1, according to a second embodiment;

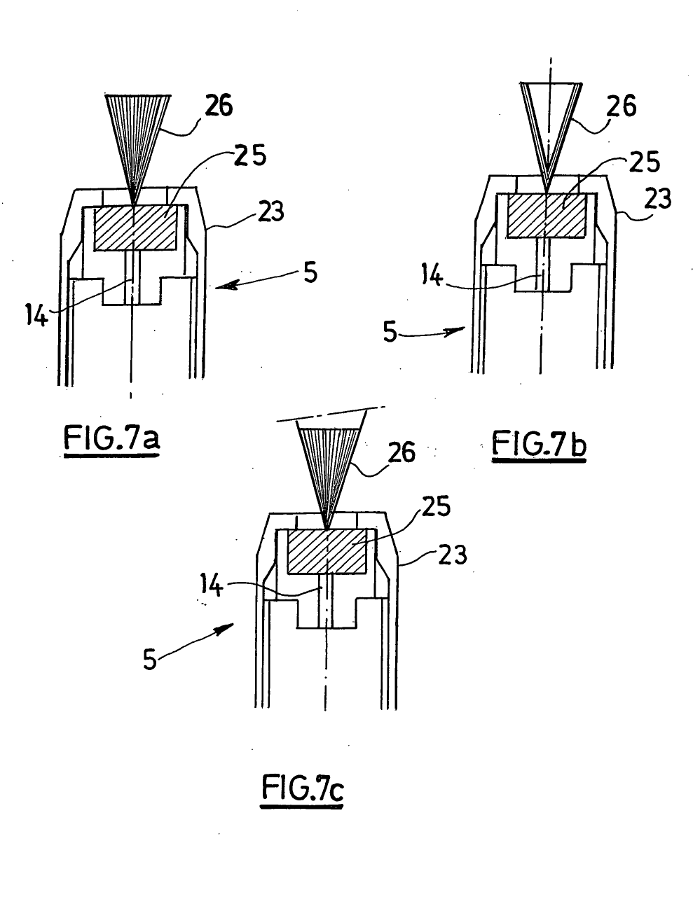

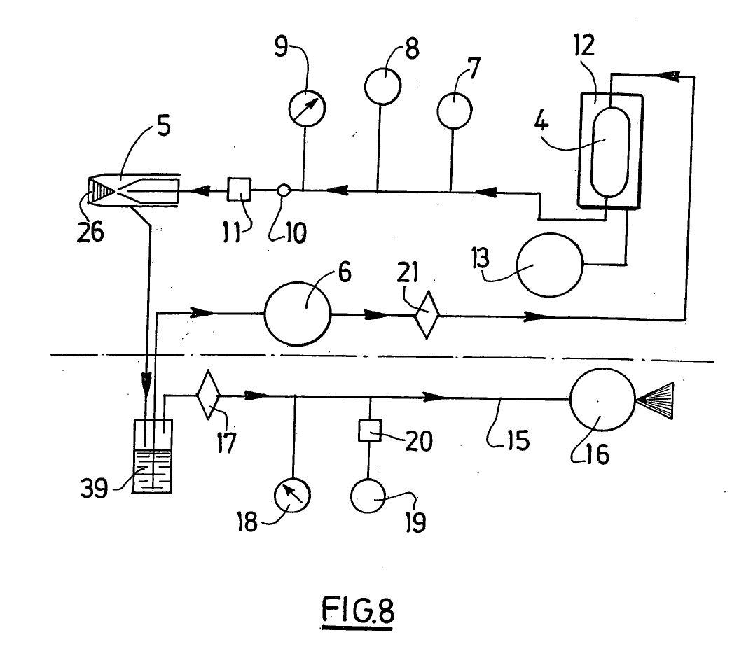

FIGS. 7a, 7b and 7c are respectively partial views in section of the hand piece of FIG. 5, showing the form of the liquid jet sprayed onto the skin, according to three variants;

FIG. 8 is a schematic view of the elements forming the device of FIG. 1, according to a variant of the second embodiment in which the device functions in closed circuit.

The method according to the invention is intended to allow penetration of active substances through the epidermis.

According to this invention, this method comprises a step consisting of spraying a pressurised liquid through a nozzle onto an area to be treated.

This method is implemented by a device 1 like the one depicted in FIG. 1. This device 1 comprises a housing 2 in which means of pressurising a liquid and means of sucking out the said liquid are disposed, a control keypad 3, bags 4 intended to receive one or more liquids, and a hand piece 5 enabling an operator to implement the said method.

According to one embodiment, depicted in FIGS. 2 and 3, the means of pressurising the liquid comprise a pump 6 which can be a piston or membrane pump, able to confer on the liquid a pressure able to vary between 5 and 70 bar. The pressurisation means are associated with means of controlling and measuring the pressure, comprising, in the direction of circulation of the liquid, a damper 7, a pressure switch 8, a pressure gauge 9, a decompressor 10 and a solenoid valve 11.

According to another embodiment, depicted in FIG. 4, the pressurisation of the liquid can be effected by an enclosure 12 in which the flexible bag 4 containing the liquid is placed, the said chamber 12 being pressurised for example by a bottle of air or nitrogen 13, or by a blower.

These pressurisation means make it possible to bring the pressurised liquid through a conduit 14 as far as the hand piece 5, by virtue of which the said liquid is sprayed onto an area of the skin.

The hand piece 5 will be described below in detail.

After having been sprayed onto the skin, the liquid is sucked up through a conduit 15. The suction means comprise a vacuum pump 16 associated with filtration means 17, as well as means of controlling and measuring the vacuum comprising a vacuum meter 18, a vacuum switch 19 and a solenoid valve 20.

FIG. 2 depicts a first embodiment of the device, in which the latter functions in open circuit. In this embodiment, the liquid is ejected after its suction into a jar 39 provided for this purpose.

According to a second embodiment, depicted in FIG. 3, the device functions in closed circuit. To this end, after suction of the liquid, the latter is put in circulation again, passing through a filter 21, so as to retain the impurities and skin waste contained in the liquid, and then the liquid is recycled, once again passing through the circuit. This arrangement has the advantage, for the same subject, of using a lesser volume of solution.

According to a variant of the second embodiment (FIG. 8), this arrangement is implemented when the circuit comprises a sterile enclosure pressurised in an enclosure 12, through which the liquid passes, so as to prevent any risk of contamination. In this variant, all the components in contact with the liquid are adapted to these sterilisation conditions.

The hand piece 5 is now described in relation to FIGS. 5 and 6.

This hand piece 5 has the conduit 14 passing through it. It consists of a body 22 whose material can withstand passing through an autoclave with a view to its sterilisation.

The hand piece 5 is provided with an on/off valve, not shown, intended to control the spraying of the liquid. In a variant, this valve can be disposed on the body of the device 1, but this arrangement has the drawback of resulting in a residual flow of the liquid remaining in the conduit 14.

The hand piece 5 is provided with an end piece 30 which can be unscrewed or detached, intended to obtain a vacuum on the skin, the said end piece 30 being rounded in shape, so as not to damage the surface of the skin to which it is applied.

The hand piece 5 also comprises a demountable or detachable nozzle holder 23, on which a spray nozzle 25 is mounted, also demountable, and sterilisable, intended to spray the pressurised liquid onto the area to be treated. Liquid arrives at this spray nozzle 25 via the conduit 14.

The spray nozzles 25 used are formed from a material of high hardness, such as tungsten carbide.

They make it possible, according to their geometry, to form jets of liquid of various shapes, various widths, or various focal distances. All variants are shown in FIGS. 7a, 7b and 7c, which show jets 26 whose shape is respectively of the triangular pencil type, or solid cone (FIG. 7a), of the hollow cone type (FIG. 7b) and of the flat jet type (FIG. 7c).

The hand piece 5 also comprises an annular suction chamber 27 connected by the conduit 15 to a collector 28, so as to allow suction of the liquid after it is sprayed onto the skin.

In the embodiment depicted in FIG. 6, the hand piece 5 comprises a body 29 and an end piece 30 mounted on the body 29, the open end of which encloses the spray nozzle 25.

In the idle position of the hand piece 5, the pressurised liquid is stopped by contact of the end 32 of the piston 36 on the piece 33.

This hand piece also comprises control means comprising a control lever 34, an electrical contactor 35 and a piston 36. These control means make it possible, when a user exerts pressure on the liquid 34, to move the end 32 of the piece 33 away, so that the orifice 37 of the conduit 14′ releases the liquid, which then fills the distribution chamber 31 and, by means of the conduit 38, supplies the spray nozzle 25.

The said control means are also arranged so as to control the functioning of the means of sucking the liquid after it is sprayed onto the skin. These suction means comprise an annular section 15′ and a cylindrical conduit 15.

The sprayed liquid comprises typically water, or saline possibly mixed with one or more additives for cosmetic, therapeutic or anaesthetic use.

One embodiment of the method according to the invention is now described.

In order to facilitate the penetration of the active substances, the method of treating the skin transepidermally can be preceded by a step of abrasion of the stratum corneum and possibly of the upper layers of the epidermis by means of abrasive products such as crushed pits, aluminium hydroxide, glass or ceramic microballs, or any other abrasive product having for example a granulometry of around 100 to 125 microns.

This step is implemented by a hand piece 5′ (FIG. 1), arranged so as to allow the abrasion of the skin by abrasive products. Thus it is possible to use quantities of additives in small proportions in the step of spraying the liquid.

The abrasion of the stratum corneum and the pressure conferred thereafter on the jet of liquid make it possible to make the products which, in cosmetic form, normally penetrate only the upper layers of the epidermis, penetrate deeply into the epidermis.

The solution intended to be sprayed onto the skin can then be prepared by injecting one or more additives, by means of a syringe or a needle, into the flexible bag 4 containing sterilised water, or saline.

It is also possible to use solutions prepared extemporaneously or products existing commercially.

Then, as soon as this injection is performed, the following step of the method is implemented. Thus the solution remains stable and the additive or additives remain soluble in solution.

The following step consists of forming a vacuum on the skin, and then the pressurisation means are activated so as to allow a spraying of the solution under pressure onto the skin of the subject, by means of the spray nozzle 25.

A safety device ensures that the pressurisation means are set in operation only when the said vacuum is formed on the skin, so as to prevent any unwanted spraying of solution.

The spraying of the said solution is carried out at a pressure such that there is no risk of breakage of the skin, whilst imposing a maximum pressure for effective penetration of the said solution.

In a particular example, the pressures used are between 10 and 25 bar, so as to allow effective penetration of the solution into the skin.

In a particular example, the flow rates used are between 100 and 250 ml/minute. When the solution is sprayed onto the face of the subject, the duration of this step is around a few minutes, the volume of solution used typically being around 1 to 2 litres.

As the said solution is sprayed, this is sucked into the suction chamber 27, with a view to its discharge through the conduit 15. The solution is then stored in a jar 39 (FIGS. 2 and 3) so as to be thrown away after its use (open-circuit operating mode) or filtered so as to be recycled (closed-circuit operating mode).

The additives used can be trace elements (zinc, silicon, selenium etc), vitamins (A, E, C, K etc), acids such as fruit acids (sugar cane, grapefruit etc), trichloracetic, kojic, hyaluronic, salicylic etc acids, mineral salts, plant extracts, or any other additive chosen according to its specific properties. These properties may result, for example, in hydrating, anti-free radical, regenerating, restructuring, exfoliating, softening, nourishing, anti-inflammatory, healing, whitening or depigmenting functions, this list of course not being exhaustive.

Other additives can also be used, such as anaesthetics.

By way of example, the following additives can be used:
muscat rose oil or grape seed oil, for their skin regenerating and restructuring properties;
horsetail extract, for its regenerating and firming action;
tocopherol, vitamin E, having anti-radical properties;
sugar cane α-hydroxyacid (AHA), for its exfoliative properties;
extract of red vine, for its anti-blotch and decongesting properties;
extract of Arnica Montana, for its anti-blotch, antiseptic and astringent properties;
hyaluronic acid, for its hydrating action;
organic silicon;
green tea, for its anti-radical and astringent properties;
extract of carrot root, rich in vitamins B1, C and E;
extract of bearberry, which contains arbutin, known for being a melanin inhibitor;
the HPS3® molecule, obtained from a purified extract of the alga Padina Pavonica, and capable of stimulating synthesis of glucose aminoglycanes, essential in skin hydration;
lidocaine, xylocaine.

1. A method for the treatment, in particular cosmetic, of an area of the skin of a human subject transepidermally, characterised in that it comprises the step consisting of spraying a pressurised liquid onto the area to be treated, by means of a spray nozzle (25).

2. A method according to claim 1, characterised by the fact that the liquid comprises water or saline.

3. A method according to claim 2, characterised by the fact that the liquid comprises at least one additive.

4. A method according to any one of claims 1 to 3, characterised by the fact that the step consisting of spraying a liquid onto the area to be treated comprises spraying at a pressure such that there is no risk of breaking the skin in the, said area, whilst imposing a maximum pressure for effective penetration of the said liquid.

5. A method according to claim 4, characterised by the fact that the pressure is between 5 and 70 bar, in particular between 10 and 25 bar.

6. A method according to claim 1, characterised in that it comprises the step consisting of sucking the sprayed liquid with a view to discharging it.

7. A method according to claim 6, characterised in that the auction step is performed by means of an annular suction chamber (27).

8. A method according to claim 1, characterised in that it comprises the steps consisting of filtering the liquid and recycling it.

9. A method according to claim 1, characterised in that the step consisting of spraying a pressurised liquid onto the area to be treated, by means of a spray nozzle (25), is preceded by a step of abrading the stratum corneum.

10. A device for treating an area of the skin of a human subject transepidermally, comprising a hand piece (5) and a spray nozzle (25) mounted on the said piece (5), characterised by the fact that it comprises means of pressuriaing a liquid, and at least one conduit (14) disposed by the pressurisation means and the spray nozzle (25).

11. A device according to claim 10, characterised by the fact that the pressurisation means comprise a pump (6).

12. A device according to claim 10, characterised by the fact that the pressurisation means comprise a bag (4) able to receive the said liquid and a chamber (12) able to receive the said bag (4).

13. A device according to any one of claims 10 to 12, characterised by the fact that it comprises an annular suction chamber (27).

14. A device according to claim 13, characterised by the fact that it comprises an end piece (30) which can be unscrewed or detached, delimiting the said chamber (27) open towards the outside, and means, for producing a vacuum in the said chamber (27).

15. A device according any one of claims 10 to 12, characterised by the fact that it comprises filtration means (21)

16. A hand piece for a device according to any one of claims 10 to 12, characterised in that it comprises control means arranged so as, in the idle position of the said hand piece, to stop the pressurised liquid by contact of the end (32) of a piston (36) on a piece (33) and, in the position of use, to move away the said end (32) of the piece (33), so as to release the liquid in order to supply the spray nozzle (25).
